(19) United States
(12) Patent Application Publication
(10) Pub. No.: US 2003/0204250 A1
(43) Pub. Date: Oct. 30, 2003

(54) Heart valve

(76) Inventor: Demetrio Bicer, Trabuco Canyon, CA (US)

Correspondence Address: HOGAN & HARTSON L.L.P., 500 S. GRAND AVENUE, SUITE 1900, LOS ANGELES, CA, 90071-2611, US

(21) Appl. No.: 10/134,568

(22) Filed: Apr. 26, 2002

(51) Int. Cl. A61F 2/24

(52) U.S. Cl. 623/2.4; 623/2.38; 623/910

(57) ABSTRACT

A novel heart valve for permanent implantation inside the human heart. The heart valve includes a valve body with an outer valve body surface. The outer valve body surface has at least one projection. This projection serves to locate at least one stiffener on the valve body after the valve has been assembled. The projection includes structure that defines at least a partial interruption through the projection in a direction perpendicular to the valve body's exterior surface. In preferred embodiments, the projection extends around less than the entire outer surface of the valve body, or multiple projections jointly extend around less than the entire outer surface of the valve body.

### BACKGROUND OF THE INVENTION

The invention provides an improved permanently prosthetic mechanical valve for permanent implantation into the heart of a human patient.

Valves of this general type are known in the art and often comprise an annular valve body configured to receive one, two, or more movable flaps or leaflets. These leaflets are movable between an open position that allows blood to flow through the valve body and a closed position that closes the valve body and prevents blood from flowing through it.

The valve body and the leaflets are generally manufactured separately and then assembled together. To assemble the leaflets into the valve body, one usually applies pressure to the outside of the valve body to deform it somewhat from its usual annular shape. One then slips the leaflets into the valve body. When the pressure is released, the valve body returns to its normal annular shape with ears or retainer tabs on the leaflets being received and held inside recesses formed in an interior surface of the valve body.

Such valves usually have a suture ring around the outside of the valve body. The suture ring is of cloth or another suitable material that allows the valve to be sutured in place in the patient's heart. The suture ring may be held in place on the valve body by a retainer ring that slides onto the valve body over the suture ring so that the suture ring is held between the valve body and the retainer ring.

Designing valves of this general type typically involves compromises between competing design considerations. It is highly advantageous, for example, to make the valve body wall as thin as possible to maximize the area of the opening through which the blood flows. To assemble the valve, moreover, the valve body needs to be somewhat flexible so that the valve body can be deformed under pressure to receive and hold the leaflets.

Once the leaflets are in place and the valve is implanted, though, the valve body needs to be sufficiently rigid so that the valve body will not deform unduly under the normal stresses placed on it by the patient's own beating heart. If the valve body deforms too much while the valve is in place, one or more of the leaflets may slip out of the valve body and float freely inside the patient. This is disastrous. Not only is the valve then largely or entirely non-functional, but the lost leaflet is must then be removed as well from the patient. Emergency surgery is then necessary, if such is even possible, to find and remove the floating leaflet and to replace the failed valve with a new one.

It is desirable therefore, to devise a mechanical heart valve whose structure is such that the valve body is sufficiently flexible during manufacturing to allow the leaflets to be installed conveniently into the valve body. The valve body will be deformed slightly for installation of the leaflets, but the valve body should then return to its original annular shape with minimal residual stress in the structure of the valve body. Conventional valve housings are generally manufactured, therefore, from materials that are flexible enough to allow for some temporary distortion of the annular valve body ring. Such materials have included, e.g., pyrolytic carbon, pyrolytic carbon-coated graphite, and titanium alloys. In deforming the annular valve body for installation of the leaflets, it is important to avoid damaging the body or introducing residual stress into the body material. Such damage or residual stress could later cause the valve to rupture or lead to another catastrophic failure of the implanted valve.

The valve body of the assembled heart valve, though, should be highly rigid so that the leaflets are held very securely in the valve body with a minimal chance of a leaflet escaping and being lost from the valve body. It is desirable moreover, to provide a structure that allows for the convenient provision of a suture ring, and moreover one that is easily and conveniently assembled, with minimum component rejection, e.g., due to broken valve bodies or residual micro-cracks in the valve body material. The assembled valve should also provide a large flow area through the valve body so that blood may flow through the valve with maximum efficiency. The invention provides a valve having these and other advantages that can be more fully understood by referring to the written descriptions in this document, and the illustrations provided herein to accompany those descriptions.

### SUMMARY OF THE INVENTION

The invention provides a novel heart valve configured for convenient assembly and permanent and secure implantation inside the heart of a living human patient. A heart valve incorporating the invention will usually include a valve body with an outer valve body surface. The outer surface of the valve body will have at least one projection on it, and this projection will serve to locate at least one stiffener on the valve body after the valve has been assembled. The projection will typically include structure that defines at least a partial interruption through the projection in a direction substantially perpendicular to the valve body's exterior surface. In preferred embodiments, the projection will extend around less than the entire outer surface of the valve body, or multiple projections may be used which jointly extend around less than the entire outer surface of the valve body.

### BRIEF DESCRIPTION OF THE DRAWINGS

FIG. 1 is a plan view showing a first implantable prosthetic heart valve that embodies the invention.

FIG. 2 is a side section view of a valve body used in the heart valve shown in FIG. 1;

FIG. 3 is a side view of the valve body shown in FIG. 2;

FIG. 4 is a side view of the heart valve of FIG. 1, fitted with stiffening rings over the valve body of FIGS. 2 and 3.

FIG. 5 is a plan view of a second valve body for an alternative heart valve that embodies the invention.

FIG. 6 is a side section view of the valve body of FIG. 5.

FIG. 7 is a plan view illustrating a valve body for a third alternative heart valve embodying the invention.

FIG. 8 is a plan view showing a valve body for a fourth alternative heart valve that embodies the invention

FIG. 9 is a side section view that illustrates a suture ring configuration usable with any of the heart valves depicted in FIGS. 1-8.

### DESCRIPTION OF THE PREFERRED EMBODIMENTS

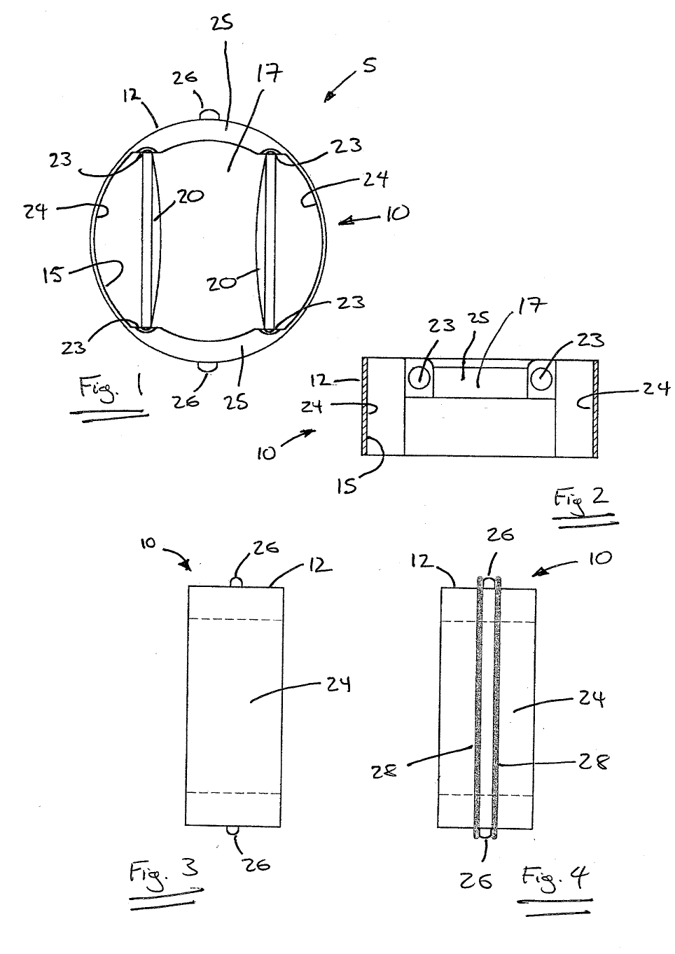

FIG. 1 is a plan view showing elements of an implantable prosthetic heart valve 5 that embodies the invention. The heart valve includes a generally annular valve body 10. The valve body includes an outer surface 12, and an inner surface 15. The inner surface defines a central flow path 17, through which blood will flow after the valve has been implanted inside the heart of a human patient.

The valve 5 of FIG. 1 includes a pair of leaflets 20. Each leaflet is supported at each end by a tab (not shown), which is received into a pivot support 23 formed on the inner surface 15 of the valve body 10. The leaflets rotate about the tabs between their open and closed positions. The leaflets are shown in this figure in the open position. In the closed position, the leaflets occlude substantially the entire flow path 17 through the valve body, thereby blocking blood flow through the valve.

The valve body 10 includes relatively thin-walled portions 24, and relatively thick-walled portions 25, with the pivot supports 23 being formed in the relatively thick-walled portions. The valve 5 includes a pair of mutually projections 26 formed on the outer surface 12 on either side of the valve body. The function and significance of these projections in the valve's overall construction are described in more detail below.

FIG. 2 is a side section view of the heart valve 5 shown in FIG. 1. This view shows clearly the relatively thin-walled portions 24 of the valve body 10, and the pivot supports 23 formed in the relatively thick-walled portions 25 to hold the tabs on each end of the leaflets 20.

FIG. 3 is a side view of the heart valve 5 depicted in FIGS. 1 and 2. This figure shows the projections 25 on either side of the outer surface 12 of the valve body 10.

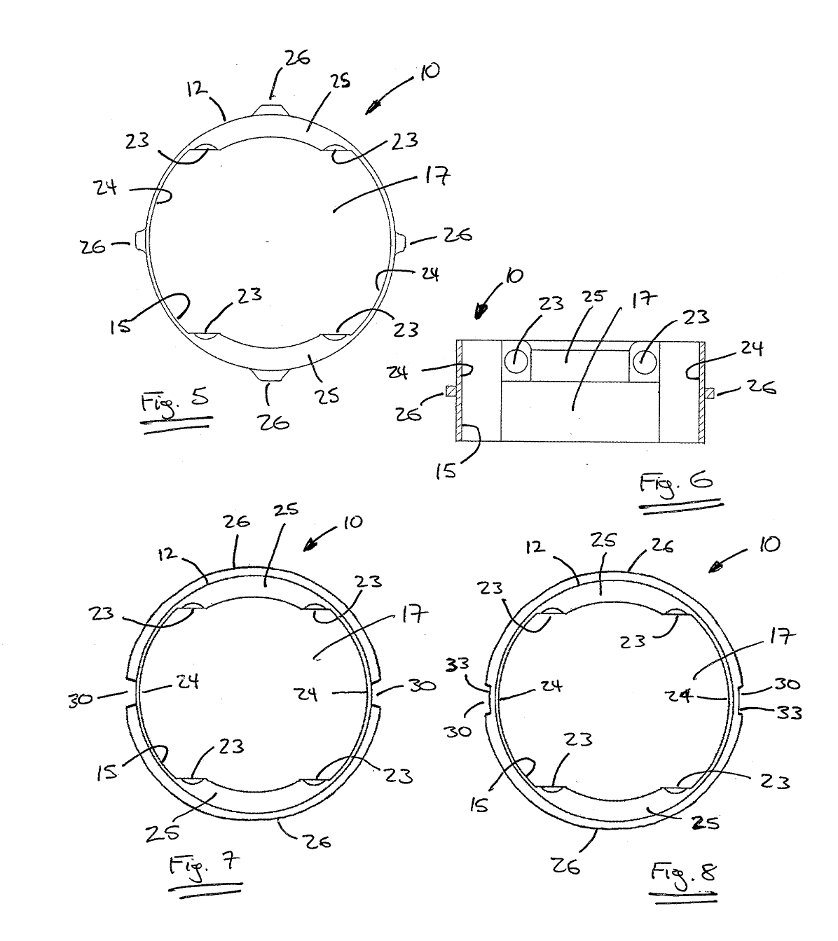

FIG. 4 is a side view identical to that of FIG. 3, but showing a pair of stiffening rings 28 slipped over the outer surface 12 of the valve body 10. As the figure indicates, the projections 26 on the valve body serve to locate the stiffening rings on the valve body.

Without the stiffening rings, the valve body 10 is relatively flexible. This is convenient in that it allows the valve body to be deformed relatively easy under pressure so that the leaflets 20 can be installed into the pivot supports 23 in the valve body. If the valve body were left in this state, though, it might be too flexible and deform under stress placed on it by the tissues of the patient's own beating heart. One or more of the leaflets might then be lost as described above, and the valve 5 fail thereby.

After the leaflets 20 are installed, the valve body 10 no longer needs to be flexible. To the contrary, it is best if the valve body is then made highly rigid and stiff. The stiffening rings 28 are slipped onto the valve body to reinforce it and make the assembled valve 5 as rigid as desired. Again, the projections 26 on the outer surface 12 of the valve body serve to locate the stiffening rings at the desired position on the valve body.

FIG. 5 is a plan view of alternative embodiment that also embodies the invention. In this valve 5, the valve body 10 has four projections 26 that are located substantially uniformly around the outer surface 12 of the valve body. As FIG. 6 indicates, these four projections once again serve to locate stiffening rings 28 on the valve body after the leaflets 20 have been assembled into the valve.

In each of the two embodiments described in this document, the projections 26 jointly extend around much less than the entire circumference of the valve body 10. For example, the embodiment shown in FIG. 1 includes two projects. Each of these two projections extends around somewhat less than 10 degrees out of the entire 360 circumference of the valve body's outer surface 12. Jointly, the two projections extend around somewhat less than 20 degrees of the valve body's circumference, or somewhat less than about 6 percent.

A single projection 26 could be provided in the form of an annular ridge extending around the entire 360 degree circumference of the valve body 10. In that case, though, the complete projection or ridge might itself decrease substantially the flexibility of the valve body. This could decrease the ease with which the valve body could be deformed for insertion of the leaflets 20 into the valve body during the valve's assembly. For this reason, it is best if the projection does not extend completely around the entire circumference of the valve body.

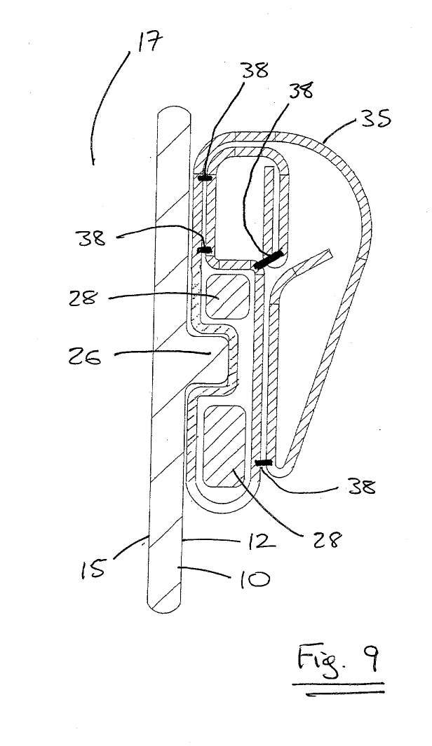

At the very least, at least one gap, if only a relatively small notch, should be provided between individual projections. FIG. 7 is a plan view illustrating a valve 5 in which two projections 26 form a nearly complete ridge around the outer surface 12 of a valve body 10. The ridge formed by the two projections is interrupted here only by a pair of notches 30, each of which overlies one of the thin wall portions 24 of the valve body. Although the ridge in this embodiment extends around nearly the entire valve body, the presence of the notches in the ridge nevertheless preserves much of the flexibility of the valve body (before the stiffening rings are installed).

In practice, the projections 26 have only to provide sufficient structure to locate the stiffening rings 28 on the valve body 10. For this reason, it will be preferred in many cases for the projections to extend around only a relatively small portion of the valve body. For example, in the valve 5 shown in FIG. 5, each of the smaller two of the four projections, which overlie the relatively thin-walled portions 24 of the valve body, extends around only somewhat less than 12 degrees of the valve body's circumference. Each of the bigger two, which are centered over the relatively thick-walled portions 25, extends around only somewhat less than 18 degrees of the valve body's entire circumference. Collectively, then, these four projections extend over about 60 degrees, or about 16 percent of the valve body's entire 360 degree circumference. This configuration allows ample flexibility in the valve body for installation of the leaflets. The structure of the projections in this embodiment is sufficient, moreover, to locate the stiffening rings 28 adequately on the assembled valve.

Various embodiments of the invention may be found useful. In most embodiments, at least one projection will be provided on the outer surface of the valve body. This projection will serve to locate one or more stiffeners, usually in the form of at least one stiffening ring, on the outer surface of the valve body after the leaflets have been installed in the valve. In preferred embodiments, the projection or projections will extend around less than the entire outer surface of the valve body, as is the case with those embodiments described above and depicted in FIGS. 1-7. This will allow positive location of the stiffener on the valve body, without interfering unduly with the flexibility of the valve body. This flexibility is helpful in installing the leaflets in the valve.

In preferred embodiments, the projection or projections will extend around only a small part of the valve body's entire outer surface, as is the case in the embodiments described above and illustrated in FIGS. 1-6. It may be thought preferable, for example, that the projections combine to extend around less than 10 percent of the valve body's entire outer surface, as is the case with the embodiment described above and shown in FIGS. 1-4. In other embodiments, the projections will combine to extend around less than about 20 percent of the valve body's entire outer surface, as is the case with the embodiment shown in FIGS. 5 and 6. Generally, at least, the projections will combine to extend around less than half (50 percent) of the valve body's entire outer surface. In some embodiments, though, the projection or projections may extend jointly around almost the entire outer surface of the valve body with only one or more small interruptions, as in the embodiment shown in FIG. 7.

In preferred embodiments one or more interruptions will be present in or between adjacent projections on the valve body's outer surface. These interruptions will generally be substantially complete, so that the projection or projections on the valve body's outer surface are completely interrupted, creating an opening in or between the projections substantially down to the surface of the valve body itself Other embodiments may include one or more interruptions in or between projections that are only partial, i.e., the interruptions may extend only partially through the projection or projections, and not fully down to the outer surface of the valve body. This is the case, for example, in the embodiment shown in FIG. 8.

As with the embodiments described above, the embodiment of FIG. 8 includes projections 26 on the outer surface 12 of the valve body 10. This embodiment, though, includes partial interruptions 33 in the projections. Put another way, the projections include structure that defines a partial interruption in the projections in a direction generally perpendicular to the surface of the valve body 10.

In this embodiment, the partial interruptions overlie the thin wall portions 24 of the valve body. As in the other embodiments, the interruptions between the projections enhance the flexibility of the projections on the valve body, and provide flexibility sufficient to allow the valve body to be deformed as the leaflets are being installed in the valve body. These projections may also serve to locate one or more stiffeners in the form of stiffening rings, for example, on the valve body when the valve is fully assembled.

The various parts of the valve may be made of materials suitable for forming the valve's parts and assembly as shown. The valve body, leaflets, and other parts including the stiffeners may be formed of, e.g., pyrolytic carbon, titanium alloys, or pure titanium. These and various other materials are amenable to formation into the required shapes according to manufacturing methods already known to those of skill in the art. These materials and others are also sufficiently durable and biocompatible for permanent implantation in the beating heart of a living human patient.

FIG. 9 is a partial side section view that shows a configuration according to which a suture ring 35 may be installed onto the heart valve 5 over the outer surface 12 of the valve body 10. In this embodiment, the suture ring is wrapped around and held in place by the stiffening rings 28, which are installed over the valve body in locations determined by the placement of a projection 26, which is provided as described above on the outer surface of the valve body.

The suture ring is installed as depicted on the valve body by first placing the suture ring over the valve body. The stiffening rings are then placed on the valve body over the suture ring, with the projections locating the stiffening rings on the valve body. The suture ring is then wrapped around the stiffening rings as shown, and joined to itself with stitches to secure the suture ring in place in the configuration shown in the figure.

The suture ring is generally formed of cloth or another suitable material. Stitches are provided through the suture ring at stitch sites 38, or elsewhere as necessary, to join parts of the suture ring to one another in order to maintain the ring's configuration on the outside of the valve. Such a suture ring can be used, as is understood by those of skill in the art, to install and anchor the valve by suturing at a suitable location inside the heart of a living human patient.

A variety of embodiments may be devised for heart valves that incorporate the invention. Many preferred embodiments will include one or more relatively small projections on the outer surface of the valve body. Together, these projections will extend around only a relatively small portion of the valve body's outer surface. These projections will generally serve to locate stiffeners or reinforcements such as stiffening rings over the valve body's outer surface after the valve has been assembled. The number of projections may vary—one, two, or more—as may the locations of the projections on the valve body's outer surface.

In other preferred embodiments, the one or more projections will extend around a considerably greater portion of the valve body's entire outer surface. In these embodiments, though, the projections will be interrupted by one or more substantially complete interruptions or gaps in or between adjacent projections, so that the projections are interrupted through substantially their entire thickness, down to outer surface of the valve body itself. Other embodiments will include projections with interruptions that are only partial and which do not extend fully through the entire thickness of the projections.

Still further embodiments may be devised by those of skill in the art, as may further modifications and additions to the modifications described more specifically above. The full scope of the invention should not be limited to the preferred and alternate embodiments described specifically herein. Rather, the full scope of the invention should be ascertained primarily by reference to the appended claims, along with the full scope of equivalents to which those claims are legally entitled.

What is claimed is:

1. A heart valve comprising:
a valve body having an outer surface;
a stiffener placed over the outer surface of the valve body; and
a projection on the outer surface of the valve body;
wherein the projection locates the stiffener on the valve body; and
wherein the projection extends around less than the entire outer surface of the valve body.

2. The heart valve of claim 1, wherein the projection extends around less than 50 percent of the entire outer surface of the valve body.

3. The heart valve of claim 2, wherein the projection extends around less than 20 percent of the entire outer surface of the valve body.

4. The heart valve of claim 3, wherein the projection extends around less than 10 percent of the entire outer surface of the valve body.

5. The heart valve of claim 1, wherein the stiffener is a stiffening ring.

6. The heart valve of claim 5, and further comprising a second stiffening ring, and wherein the first and second stiffening rings are configured for placement on opposite sides of the projection.

7. The heart valve of claim 1, and further comprising a second stiffener, and wherein the first and second stiffeners are configured for placement on opposite sides of the projection.

8. The heart valve of claim 1, and further comprising a suture ring, wherein the suture ring is retained on the valve body by the stiffener.

9. The heart valve of claim 8, and wherein the suture ring is wrapped around the stiffener.

10. A heart valve comprising:
a valve body having an outer surface;
a stiffener placed over the outer surface of the valve body; and
first and second projections on the outer surface of the valve body;
wherein the first and second projections locate the stiffener on the valve body; and
wherein the first and second projections together extend around less than the entire surface of the valve body.

11. The heart valve of claim 10, wherein the projections on the outer surface of the valve body together extend around less than 50 percent of the entire outer surface of the valve body.

12. The heart valve of claim 11, wherein the projections on the outer surface of the valve body together extend around less than 20 percent of the entire outer surface of the valve body.

13. The heart valve of claim 12, wherein the projections on the outer surface of the valve body together extend around less than 10 percent of the entire outer surface of the valve body.

14. The heart valve of claim 10, wherein the stiffener is a stiffening ring.

15. The heart valve of claim 14, and further comprising a second stiffening ring, and wherein the first and second stiffening rings are configured for placement on opposite sides of the projection.

16. The heart valve of claim 10, and further comprising a second stiffener, and wherein the first and second stiffeners are configured for placement on opposite sides of the projection.

17. The heart valve of claim 10, and further comprising a suture ring, wherein the suture ring is retained on the valve body by the stiffener.

18. The heart valve of claim 17, and wherein the suture ring is wrapped around the stiffener.

19. A heart valve comprising:
a valve body having an outer surface;
a stiffener placed over the outer surface of the valve body; and
a projection on the outer surface of the valve body;
wherein the projection locates the stiffener on the valve body; and
wherein the projection includes structure defining at least a partial interruption through the projection in a direction substantially perpendicular to the exterior surface of the valve body.

20. The heart valve of claim 19, wherein the projection includes structure defining at least a partial interruption through the projection in a direction substantially perpendicular to the exterior surface of the valve body so that the uninterrupted portion of the projection extends around less than 50 percent of the valve body's entire outer surface.

21. The heart valve of claim 20, wherein the uninterrupted portion of the projection extends around less than 20 percent of the valve body's entire outer surface.

22. The heart valve of claim 21, wherein the uninterrupted portion of the projection extends around less than 10 percent of the valve body's entire outer surface.

23. The heart valve of claim 19, wherein the stiffener is a stiffening ring.

24. The heart valve of claim 23, and further comprising a second stiffening ring, and wherein the first and second stiffening rings are configured for placement on opposite sides of the projection.

25. The heart valve of claim 19, and further comprising a second stiffener, and wherein the first and second stiffeners are configured for placement on opposite sides of the projection.

26. The heart valve of claim 19:
wherein the valve body includes relatively thin wall sections and relatively thick wall sections; and
wherein the interruption through the projection is located over a relatively thin wall section of the valve body.

27. The heart valve of claim 19, and further comprising a suture ring, wherein the suture ring is retained on the valve body by the stiffener.

28. The heart valve of claim 27, and wherein the suture ring is wrapped around the stiffener.

29. A method for making a heart valve, the method comprising:
providing a valve body with an exterior surface and a projection that extends around less than the entire exterior surface of the valve body;
placing a stiffener over the exterior surface of the valve body;
using the projection to locate the stiffener on the valve body.

30. The method of claim 29, and wherein providing a valve body with an exterior surface and a projection that extends around less than the entire exterior surface of the valve body, includes providing a valve body with a projection that extends around less than 50 percent of the valve body's entire exterior surface.

31. The method of claim 30, and wherein providing a valve body with a projection that extends around less than 50 percent of the valve body's entire exterior surface includes providing a valve body with a projection that extends around less than 20 percent of the valve boy's entire exterior surface.

32. The method of claim 31, and wherein providing a valve body with a projection that extends around less than 20 percent of the valve body's entire exterior surface includes providing a valve body with a projection that extends around less than 10 percent of the valve boy's entire exterior surface.

33. The method of claim 29, and further comprising:
installing at least one leaflet into the valve body before the stiffener is placed over the exterior surface of the valve body.

34. The method of claim 29, and further comprising placing a suture ring over the valve body.

35. The method of claim 29, and further comprising:
placing a suture ring over the valve body before the stiffener is placed over the exterior surface of the valve body.

36. The method of claim 34, and further comprising:
wrapping the suture ring over the stiffener after the stiffener is placed over the exterior surface of the valve body.

37. The method of claim 36, and further comprising:
joining the suture ring to itself to fix the suture ring in place on the valve body.

38. The method of claim 37, and wherein joining the suture ring to itself comprises stitching the suture ring to itself.

39. A method for making a heart valve, the method comprising:
providing a valve body with an exterior surface and first and second projections that together extend around less than the entire exterior surface of the valve body;
placing a stiffener over the exterior surface of the valve body;
using the projections to locate the stiffener on the valve body.

40. The method of claim 39, and wherein providing a valve body with an exterior surface and first and second projections that together extend around less than the entire exterior surface of the valve body includes providing projections that together extend around less than 50 percent of the valve body's entire exterior surface.

41. The method of claim 40, and wherein providing projections that together extend around less than 50 percent of the valve body's entire exterior surface includes providing projections that together extend around less than 20 percent of the valve body's entire exterior surface.

42. The method of claim 41, and wherein providing projections that together extend around less than 20 percent of the valve body's entire exterior surface includes providing projections that together extend around less than 10 percent of the valve body's entire exterior surface.

43. The method of claim 39, and further comprising:
installing at least one leaflet into the valve body before the stiffener is placed over the exterior surface of the valve body.

44. The method of claim 39, and further comprising placing a suture ring over the valve body.

45. The method of claim 39, and further comprising:
placing a suture ring over the valve body before the stiffener is placed over the exterior surface of the valve body.

46. The method of claim 44, and further comprising:
wrapping the suture ring over the stiffener after the stiffener is placed over the exterior surface of the valve body.

47. The method of claim 46, and further comprising:
joining the suture ring to itself to fix the suture ring in place on the valve body.

48. The method of claim 47, and wherein joining the suture ring to itself comprises stitching the suture ring to itself.

49. A method for making a heart valve, the method comprising:
providing a valve body with an exterior surface and a projection on the exterior surface, wherein the projection includes structure defining at least a partial interruption through the projection in a direction substantially perpendicular to the exterior surface of the valve body;
placing a stiffener over the exterior surface of the valve body;
using the projection to locate the stiffener on the valve body.

50. The method of claim 49, and wherein providing a valve body with an exterior surface and a projection on the exterior surface includes providing a valve body with a projection whose uninterrupted portion extends around less than 50 percent of the valve body's entire exterior surface.

51. The method of claim 50, and wherein providing a valve body with a projection whose uninterrupted portion extends around less than 50 percent of the valve body's entire exterior surface includes providing a valve body with a projection whose uninterrupted portion extends around less than 20 percent of the valve body's entire exterior surface.

52. The method of claim 51, and wherein providing a valve body with a projection whose uninterrupted portion extends around less than 20 percent of the valve body's entire exterior surface includes providing a valve body with a projection whose uninterrupted portion extends around less than 10 percent of the valve body's entire exterior surface.

53. The method of claim 49, and further comprising:
installing at least one leaflet into the valve body before the stiffener is placed over the exterior surface of the valve body.

54. The method of claim 49, and further comprising placing a suture ring over the valve body.

55. The method of claim 49, and further comprising:
placing a suture ring over the valve body before the stiffener is placed over the exterior surface of the valve body.

56. The method of claim 54, and further comprising:
wrapping the suture ring over the stiffener after the stiffener is placed over the exterior surface of the valve body.

57. The method of claim 56, and further comprising:
joining the suture ring to itself to fix the suture ring in place on the valve body.

58. The method of claim 57, and wherein joining the suture ring to itself comprises stitching the suture ring to itself.
